(19) United States
(12) Patent Application Publication
(10) Pub. No.: US 2022/0031882 A1
(43) Pub. Date: Feb. 3, 2022

(54) AUTOMATED MOBILE ROBOT WITH UVC LIGHTS FOR DISINFECTING A FACILITY

(71) Applicant: BOTLEY LLC, Highland, MI (US)

(72) Inventors: David Dreffs, Highland, MI (US); Robert Dow, Highland, MI (US)

(21) Appl. No.: 17/391,217

(22) Filed: Aug. 2, 2021

Related U.S. Application Data
(60) Provisional application No. 63/059,834, filed on Jul. 31, 2020.

(30) Foreign Application Priority Data
Aug. 25, 2020 (CA) 3091069

(51) Int. Cl. A61L 2/10; B25J 15/00; B25J 19/00; B25J 5/00; B25J 9/16; G05D 1/02

(52) U.S. Cl. CPC A61L 2/10; B25J 15/0019; B25J 19/0025; B25J 5/007; A61L 2202/25; G05D 1/0212; A61L 2202/11; A61L 2202/14; A61L 2202/16; B25J 9/1679

(57) ABSTRACT

An automated mobile robot includes a housing, an articulated arm that has at least one UVC light thereon, an actuator that is operable to move the arm between a retracted position and an extended position, a drive unit that is operable to move the housing, a position sensor that is operable to generate position signals indicative of an instant position, a computer, and a battery connected to the actuator, the drive unit, the UVC light, and the computer. The computer is operably coupled to the actuator, the position sensor, the UVC light, and the drive unit. The computer has a predetermined disinfection route and is configured to operate the actuator to move the arm, activate and deactivate the UVC light, and operate the drive unit to move the housing along the predetermined disinfection route based on the instant position and a desired position in the predetermined disinfection route.

### BACKGROUND

Large facilities, including medical facilities, are often periodically sterilized with disinfectant fluids to minimize the spread of viruses and bacteria to individuals in the facilities. However different facilities and different rooms in the facilities may require different types of sterilizing fluids to be used therein which can be difficult to manage logistically. Further, the sterilization of large areas in the facilities is labor-intensive and expensive.

The inventors herein have recognized a need for an automated mobile robot with UVC lights that minimizes and/or eliminates the above-mentioned problem.

### SUMMARY

An automated mobile robot includes a housing, an articulated arm that has at least one UVC light thereon, an actuator that is operable to move the arm between a retracted position and an extended position, a drive unit that is operable to move the housing, a position sensor that is operable to generate position signals indicative of an instant position, a computer, and a battery connected to the actuator, the drive unit, the UVC light, and the computer. The computer is operably coupled to the actuator, the position sensor, the UVC light, and the drive unit. The computer has a predetermined disinfection route and is configured to operate the actuator to move the arm, activate and deactivate the UVC light, and operate the drive unit to move the housing along the predetermined disinfection route based on the instant position and a desired position in the predetermined disinfection route. In a further example, an automated mobile robot for disinfecting a facility is provided. The automated mobile robot includes a housing having at least first and second sides. The The automated mobile robot further includes a first extension arm having a first plurality of UVC lights coupled thereto. The first extension arm is coupled to an actuator in the housing. The actuator extends the first extension arm outwardly from the first side of the housing to move the first extension arm from a retracted position to a fully extended position thereof. The automated mobile robot further includes a second extension arm having a second plurality of UVC lights coupled thereto. The second extension arm is coupled to the actuator in the housing. The actuator extends the second extension arm outwardly from the second side of the housing to move the second extension arm from a retracted position to an fully extended position thereof. The automated mobile robot further includes a position sensor on the housing that generates position signals indicating a position of the housing in a facility. The automated mobile robot further includes a drive unit coupled to the housing that moves the housing to predetermined locations based on commands from a computer. The computer is operably coupled to the actuator, the position sensor, and the drive unit. The computer has a predetermined disinfection route for the facility stored therein. The computer induces the actuator to extend the first extension arm to the fully extended position thereof and to extend the second extension arm to the fully extended position thereof and to active the first and second plurality of UVC lights. The computer controls the drive unit to induce the drive unit to move the housing along the predetermined disinfection route in the facility.

### BRIEF DESCRIPTION OF THE DRAWINGS

FIG. 1 is an isometric view of an automated mobile robot in accordance with an exemplary embodiment;

FIG. 2 is a first side view of the automated mobile robot of FIG. 1;

FIG. 3 is a second side view of the automated mobile robot of FIG. 1;

FIG. 4 is a top view of the automated mobile robot of FIG. 1;

FIG. 5 is a front view of the automated mobile robot of FIG. 1;

FIG. 6 is a bottom view of the automated mobile robot of FIG. 1;

FIG. 7 is a rear view of the automated mobile robot of FIG. 1;

FIGS. 8, 9 and 10 are electrical wiring diagrams of a circuit utilized in the automated mobile robot of FIG. 1;

FIG. 11 is a flowchart of a method of controlling the automated mobile robot of FIG. 1.

### DETAILED DESCRIPTION

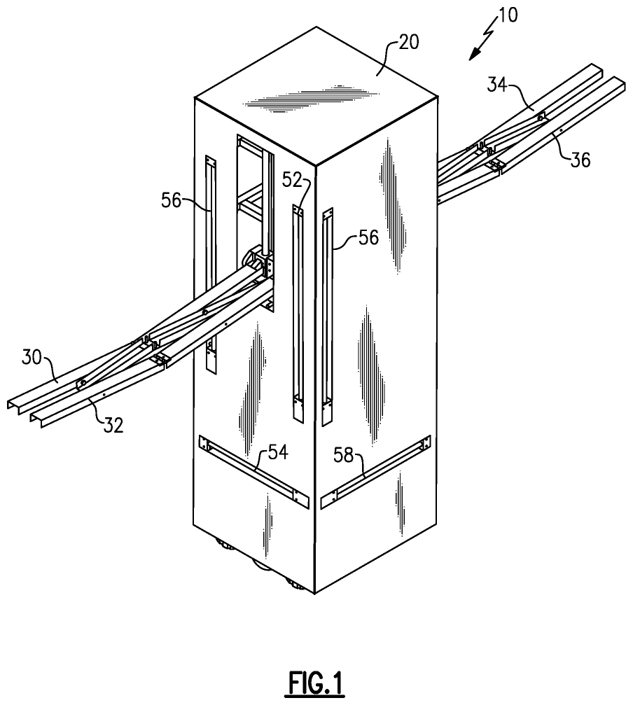

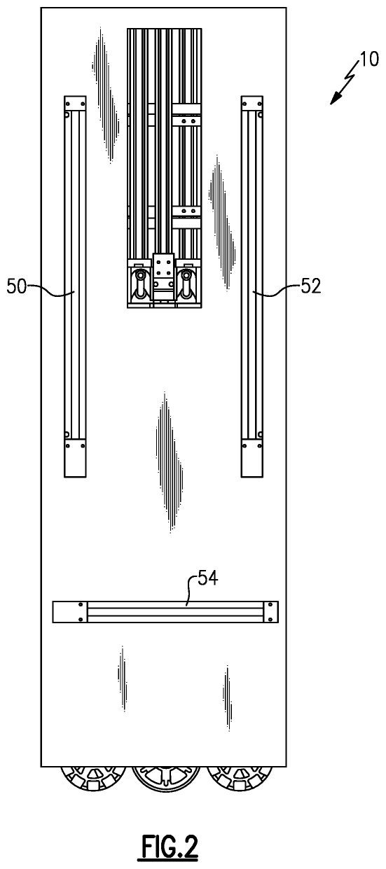

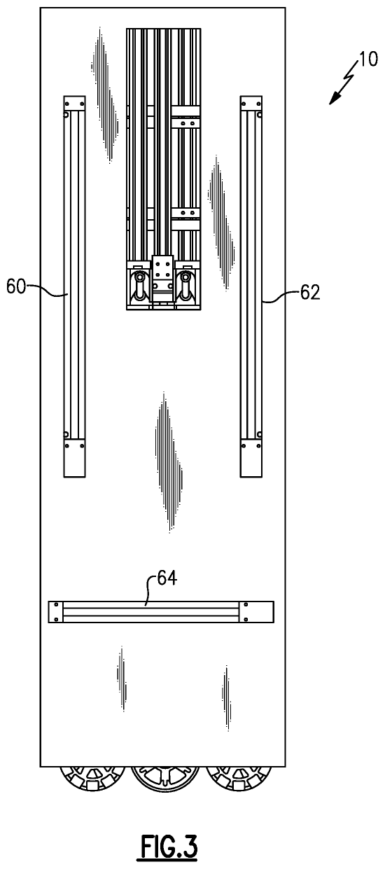

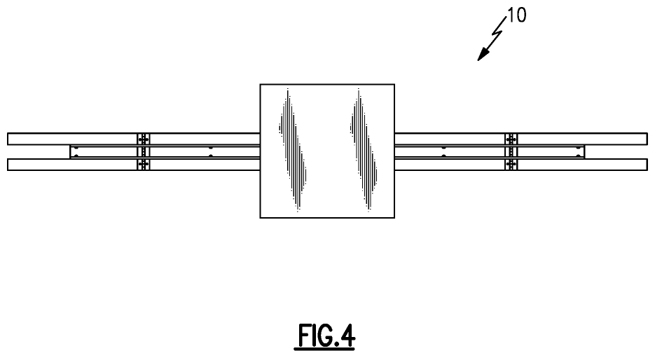

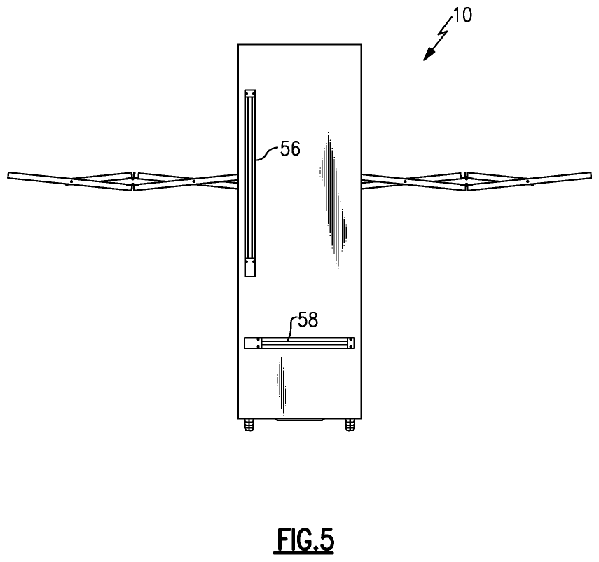

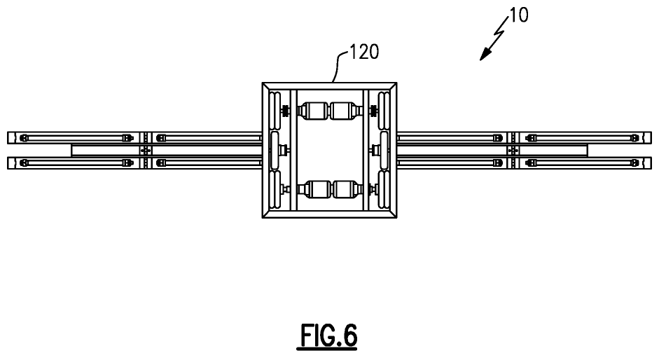

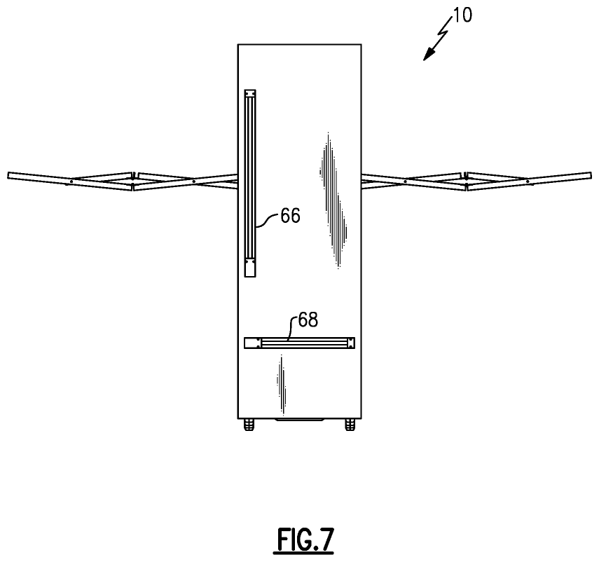

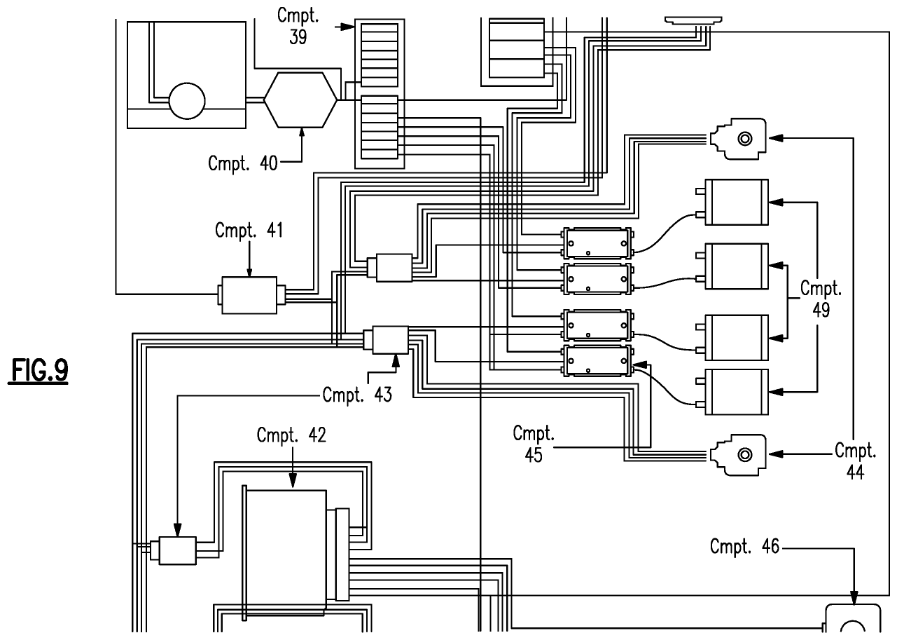

Referring to FIGS. 1-9, the automated mobile robot 10 is provided to be remotely controlled and/or to operate autonomously to disinfect facilities, such as medical facilities. In an exemplary embodiment, the automated mobile robot 10 includes a housing 20, extension arms 30, 32, 34, 36 with UVC lights, and UVC lights 50, 52, 54, 56, 58, 60, 62, 64, 66 and 68, an actuator device 90, a position sensor 90, a battery 110, a drive unit 120, and a control system 120.

The housing 20 holds the remaining components of the robot 10 therein.

The extension arms 30, 32, 34, 36 are coupled to an actuator that moves the extension arms 30, 32, 34, 36 from a retracted position to a full-extended operational position, and vice-versa in response to control signals from the control system 120. Further, the UVC lights on the extension arms 30, 32, 34, 36 are activated in response to control signals from the control system 120.

The position sensor 90 determines a position of the automated mobile robot 10. In an exemplary embodiment, the position sensor 90 is a Lidar position sensor.

The UVC lights 50, 52, 54, 56, 58, 60, 62, 64, 66 and 68 are used to disinfect a facility using ultraviolet light at a predetermined intensity and a predetermined frequency range to kill viruses and/or bacteria. The UVC lights 50, 52, 54, 56, 58, 60, 62, 64, 66 and 68 are activated in response to control signals from the control system 150.

The battery 110 provides electrical power to the control system 150, and the drive unit 120, and the UVC lights. The battery 110 is rechargeable.

The drive unit 120 is provided to move the housing 20 along a predetermined path that is determined by the control system 150. The drive unit 120 includes at least four motors and six drive wheels.

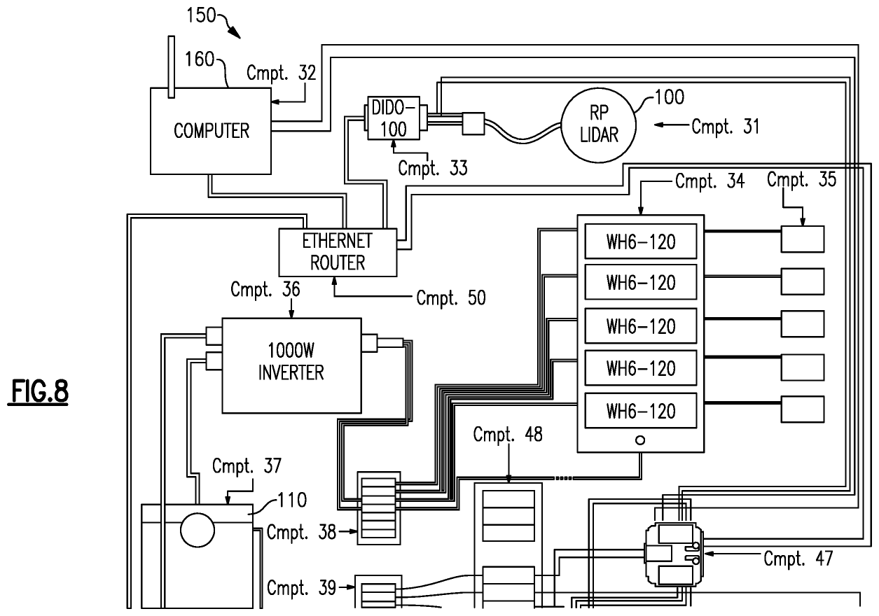

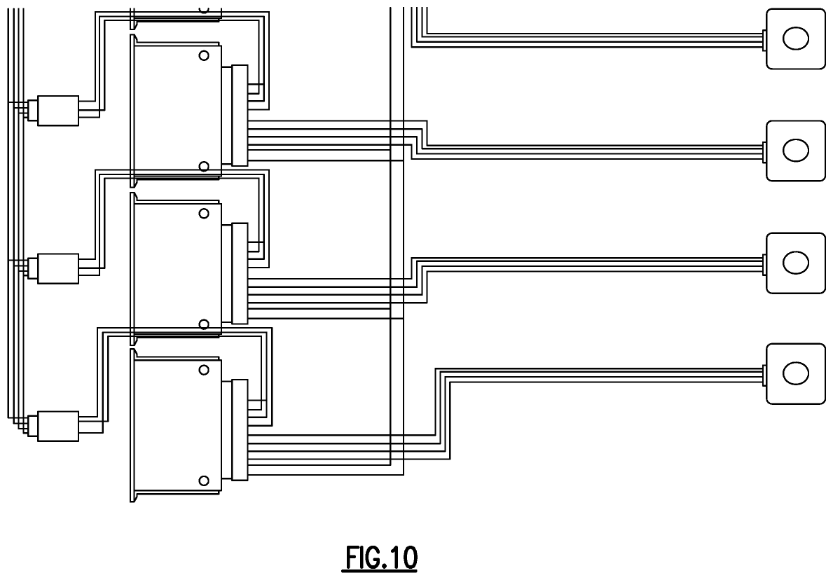

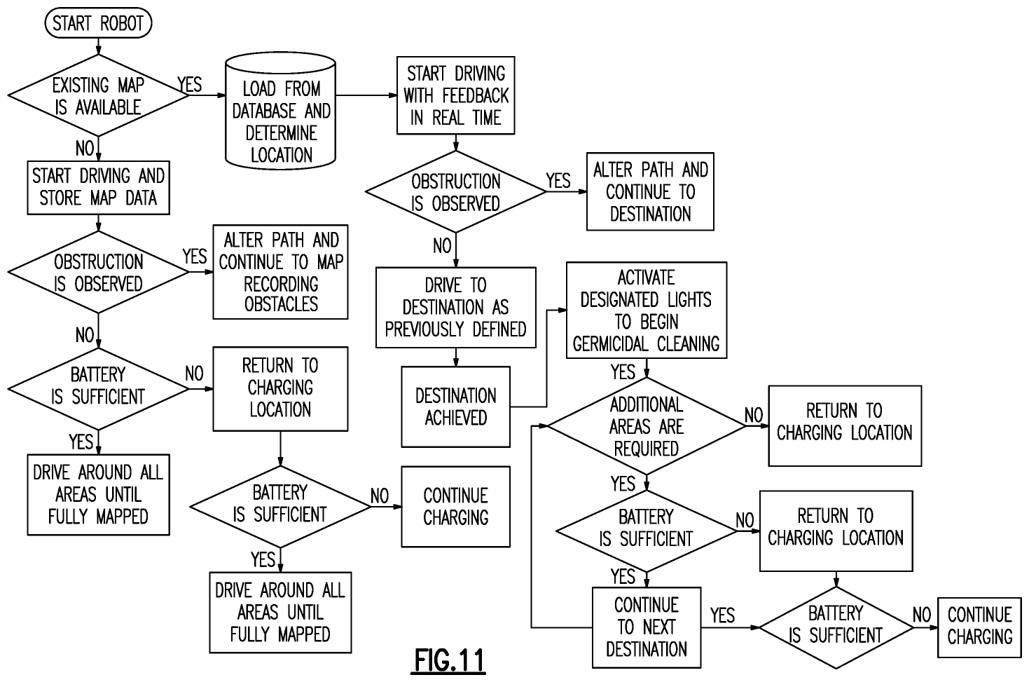

The control system 150 includes a computer 160 and is illustrated in the FIGS. 8-10. The control system 150 controls the operation of the automated mobile robot, according to the method described in FIG. 10. In particular, the computer 160 controls the operation of the direct-current motor on the basis of the measured actual position and the desired nominal position of the automated mobile robot. In the simplest case, an appropriate encoder can be fitted to the wheels themselves, the encoder measures the revolution of the wheels and emits appropriate data to the computer 160. In addition, the computer 160 may provide further data inputs and outputs, for example in order to allow switches or sensor data to be read in or display elements to be controlled. Such additional functionalities can easily be achieved by a control program which runs on the computer 160.

In an alternative embodiment, the automated mobile robot 10 may be equipped with an autonomous position transmitter, which uses a specific position transmitter wheel to record the distance traveled, largely without slip, and makes this available as position data via an encoder unit which is accommodated in the chassis. The position sensor can be fitted to a suitable point on the automated mobile robot by a universal mounting element. The computer 160 is designed such that it can read and process or pass on these additional signals without major complexity. In addition to drastically reducing positioning error, this position sensor therefore also makes it possible to implement slip monitoring and to provide an appropriate warning to the superordinate program or the operator.

In the present case, a (passive) steering roller is mounted underneath the housing 10. The steering roller has two wheels which are arranged parallel and are mounted via a rotating bearing such that they can rotate about a vertical axis. A roller such as this can advantageously be used for steering the automated mobile robot. Another steering option is provided by differentially driving to the two drive modules.

The automated mobile robot 10 can provide directional UVC (or optionally laser light) for sterilization and can generate ions for sterilization. Optionally, the system can generate ions and direct to surface or blanket ion emissions for surface disinfection and sterilization. It is envisioned the robot 10 can have sonar, IR and laser range finding navigation transceivers which can map room and surfaces, generate topographical 3D map for robot navigation and surface sterilization. Additionally, the robot 10 can provide sensing devices such as a spectrometer to measure airborne bacteria, molds and viruses to apply unidirectional U.V. and laser sterilization. Optionally, the robot 10 can utilize optical or infra-red sensors to enable automatic safety shutoff upon encountering a human or a human shape. Additionally, the robot 10 can have pre-defined routines which allow for the disinfection of medical devices. This shutoff system can also optionally detect the remote opening of a door into the facility.

The robot 10 can include processors which allow for adaptive learning. Optionally, the robot 10 can be wirelessly controlled by an operator and can include an imaging device such as a color stereo and 3D cameras to allow an operator to remotely disinfect an area. Optionally, the wireless control, communication and data transfer can occur from one robot to another to teach one another.

The computer 160 can be used for direct access or web-based control of the robot 10. The robot 10 can respond to voice commands and control, can be speech capable. Navigation can occur using a pre-mapped area. Also, motion sensors can be utilized to track object movement within the disinfecting area.

The robot 10 can be utilized in medical facilities and food processing environments for example. Internal ethernet communication can be used to communicate between various modules. More than one robot 10 can dock together to transfer power or between robots. Optionally, the robot 10 can incorporate sensors which will allow the robot 10 to avoid obstacles and allow the robot 10 to be controlled by smart phone applications. In alternative embodiment, the robot 10 can detect the surroundings thereto using a laser 3D depth range finder, or a 360 degree vision with miniature cameras connected to emulate panoramic-vision, and/or a bar code reader.

The automated robot 10 can be utilized to disinfect regions under hospital beds and surgical tables. UV light and laser emitters directed at the underside of beds, surgical tables, hospital furniture, equipment and building structures. Bottom of vehicle has UV light and laser emitters directed at floor. Circumference of robot has UV light and laser emitters for side way projection of disinfecting light. The robot 10 can dispense Luminal to detect the presence of blood and blood products. Further, in an alternative embodiment, the robot 10 can have forward, rear and upward looking cameras, and mapping and avoidance sensors. The robot 10 can be remotely controlled by wireless or wired hand-held controllers, web applications, IR, laser over fiber optic, ethernet etc.

The facilities that are being disinfected can include tracking and locating beacons to facilitate movement of the automated mobile robot 10. Optionally, the automated mobile robot 10 can utilize GPS coordinates to determine a position thereof. Further, a room can include self-docking in a recharging dock station for the robot 10. The enclosed docking station can be used for self-decontamination and self-maintenance, and sense internal status. For example, if a low battery is detected, the robot 10 can self-dock to be recharged.

While the claimed invention has been described in detail in connection with only a limited number of embodiments, it should be readily understood that the invention is not limited to such disclosed embodiments. Rather, the claimed invention can be modified to incorporate any number of variations, alterations, substitutions or equivalent arrangements not heretofore described, but which are commensurate with the spirit and scope of the invention. Additionally, while various embodiments of the claimed invention have been described, it is to be understood that aspects of the invention may include only some of the described embodiments. Accordingly, the claimed invention is not to be seen as limited by the foregoing description.

What is claimed is:

1. An automated mobile robot, comprising:
a housing;
at least one articulated arm extendable from the housing and having at least one UVC light thereon;
an actuator coupled with the arm and operable to move the arm between a retracted position and an extended position;
a drive unit operable to move the housing;
a position sensor operable to generate position signals indicative of an instant position of the housing;
a computer; and
a battery connected to the actuator, the drive unit, the UVC light, and the computer, wherein
the computer is operably coupled to the actuator, the position sensor, the UVC light, and the drive unit, the computer having a predetermined disinfection route stored therein, the computer configured to operate the actuator to move the arm, activate and deactivate the UVC light, and operate the drive unit to move the housing along the predetermined disinfection route based on the instant position and a desired position in the predetermined disinfection route.

2. The automated mobile robot as recited in claim 1, further comprising a steering roller mounted under the housing, the steering roller including two parallel wheels mounted on a rotating bearing such that the two parallel wheels are rotatable about a vertical axis.

3. The automated mobile robot as recited in claim 1, wherein the housing is moveable on a plurality of drive wheels, and further comprising an encoder on at least one of the drive wheels, the encoder operable to generate signals indicative of revolution of the at least one of the drive wheels.

4. The automated mobile robot as recited in claim 1, wherein the housing includes a position transmitter wheel operable to measure a distance travelled by the housing.

5. The automated mobile robot as recited in claim 1, further comprising a camera on the housing.

6. The automated mobile robot as recited in claim 1, further comprising an optical or infrared sensor connected with the computer, and the computer is configured to shut off the at least one UVC light responsive to detection of a human via the optical or infrared sensor.

7. The automated mobile robot as recited in claim 1, further comprising a docking station that is configured to recharge the battery.

8. The automated mobile robot as recited in claim 7, wherein the computer is configured to move the housing to the docking station responsive to a low power level of the battery.

9. The automated mobile robot as recited in claim 1, wherein the at least one articulated arm includes a scissor extension.

10. The automated mobile robot as recited in claim 9, wherein the scissor extension includes four pivotably connected links, and the at least one UVC light includes four UVC lights mounted, respectively, on the four pivotably connected links.

11. The automated mobile robot as recited in claim 1, wherein the position sensor is a Lidar position sensor.

12. An automated mobile robot, comprising:
a vertically upstanding housing defining first and second opposed sides;
first and second articulated arms extendable from, respectively, the first and second opposed sides, each of the first and second articulated arms having at least one UVC light;
first and second vertically-oriented UVC lights disposed on, respectively the first and second opposed sides laterally adjacent the first and second articulated arms;
first and second horizontally-oriented UVC lights disposed on, respectively, the first and second opposed sides below the first and second articulated arms;
an actuator coupled with the articulated arms;
a drive unit operable to move the housing;
a position sensor;
a battery; and
a computer operably coupled to the actuator, the position sensor, the at least one UVC light, the vertically-oriented UVC lights, the horizontally-oriented UVC lights, and the drive unit, the computer being configured to move the housing via the drive unit along a predetermined disinfection route.

13. The automated mobile robot as recited in claim 12, further comprising third and fourth vertically-oriented UVC lights disposed on, respectively the first and second opposed sides laterally adjacent the first and second articulated arms such that the first articulated arm is between the first vertically-oriented UVC light and the third vertically-oriented UVC light and the second articulated arm is between the second vertically-oriented UVC light and the fourth vertically-oriented UVC light.

14. The automated mobile robot as recited in claim 12, wherein the first and second vertically-oriented UVC lights are vertically coextensive.

15. The automated mobile robot as recited in claim 12, wherein the at least one articulated arm includes a scissor extension.

16. The automated mobile robot as recited in claim 12, wherein the vertically upstanding housing defines third and fourth opposed sides that join the first and second opposed sides, each of the third and fourth opposed sides having at least two additional UVC lights.

17. The automated mobile robot as recited in claim 12, wherein the housing is moveable on drive wheels that are rotatable about respective drive axes that are parallel to each other, and the first and second articulated arms are extendable in an extension direction that is parallel to the drive axes.
